(19) United States
(12) Patent Application Publication
(10) Pub. No.: US 2025/0050544 A1
(43) Pub. Date: Feb. 13, 2025

(54) Caking-resistant neopentyl glycol pellets and method for producing caking-resistant neopentyl glycol pellets

(71) Applicant: OQ CHEMICALS GMBH, Monheim am Rhein (DE)

(72) Inventors: Horst LANGE, Bochum (DE); Julia ZIMMERER, Köln (DE)

(21) Appl. No.: 18/721,623

(22) PCT Filed: Dec. 14, 2022
(86) PCT No.: PCT/EP2022/085738

§ 371 Date: Jun. 18, 2024

(30) Foreign Application Priority Data
Dec. 23, 2021 (EP) 21217297.7

(51) Int. Cl. B29B 9/10; B29K 33/04

(52) U.S. Cl. CPC B29B 9/10; B29K 2033/04; B29K 2995/0063

(57) ABSTRACT

The present subject matter relates to a process for producing neopentyl glycol compacts, the process including at least the process steps of providing a fill of NPG flakes and compressing the fill in a mold to form a compact, wherein the compressing is performed at a pressure greater than or equal to 0.5 MPa and less than or equal to 7.5 MPa. Furthermore, the present subject matter relates to NPG compacts.

The present invention relates to a process for making neopentyl glycol compacts, the process comprising at least the steps of:
a) providing a fill of NPG flakes;
b) compressing the fill in a mold to form a compact, wherein the compressing is performed at a pressure of greater than or equal to 0.5 MPa and less than or equal to 7.5 MPa.

Furthermore, the present invention relates to NPG compacts.

Neopentyl glycol (NPG, 2,2-dimethylpropane-1,3-diol) is an important diol that is used in large quantities, for example, in the production of polyesters and polyurethanes. The industrial production of NPG usually starts from isobutylaldehyde, which is reacted with formaldehyde and the reaction product is then catalytically hydrogenated. The NPG is a hygroscopic, crystalline compound, which has a melting point of approximately 129° C. For reasons of cost and practicality, the diol is offered in the form of smaller flakes, which are produced by solidification from an NPG melt with the aid of a crystallization or cooling belt and subsequent breaking into individual, more or less irregular platelets. The NPG flakes are then conveniently packaged and shipped in big bags of greater than or equal to 250 kg or as bagged goods of greater than or equal to 25 kg each. Based on the hygroscopic and complex thermodynamic phase properties of the NPG, as a function of the specific storage and transport conditions present, the flakes may agglomerate overtime in the form of larger aggregates. The unregulated agglomeration can lead to the formation of very large, compact NPG clumps, which can cause the ability of the product to flow freely to approach zero. This effect can go so far as to cause the entire contents of a big bag to cake in large volumes, making it impossible to efficiently empty the big bag for further processing. In order to use the product in standard production processes, a time-consuming and expensive manual comminution to the again free-flowing product must be carried out before further processing.

This unfavorable condition leads to major disruptions in the production process and usually to complaints to the manufacturer.

The patent literature also contains different approaches to packaging and improving the NPG caking or packaging problem.

For example, U.S. Pat. No. 4,435,603A teaches the addition of tertiary amines in concentrations of 0.25-0.5 wt. % as anti-caking agents in the production of polyol flakes, in particular neopentyl glycol flakes. However, experience has shown that even the addition of such anti-caking agents does not reliably prevent the formation of material caking, coarse lumps and clumps, or large-volume product caking, especially during the storage of palletized bags or is big bags.

For example, DE 3 522 359 A1 describes a process for making up organic materials that are crystalline under normal conditions, in which the materials in powdered and/or molten state are prepared in a self-cleaning twin-screw machine with screw shafts rotating in the same direction, are discharged through at least one constricted passage into a zone of low pressure, cooled and broken into particles, characterized in that the materials are heated during discharge through the constricted passage to form a melt film.

Furthermore, EP 0 829 298 A2 discloses a process for the production of hydroxypivalic acid neopentyl glycol ester granules by applying a hydroxypivalic acid neopentyl glycol ester melt to a cooling surface on which the melt solidifies, characterized in that the melt contains at least 3% by weight, based on the total amount of hydroxypivalic acid neopentyl glycol ester, of hydroxypivalic acid neopentyl glycol ester crystals.

EP 1 268 378 B1 also describes a process for making up neopentyl glycol by cooling, crystallizing and crushing a neopentyl glycol melt and then packaging the neopentyl glycol particles thus obtained in storage or transport containers. In this process, at the beginning of cooling, the melt is cooled for at least 1/10 minute without using a coolant or using a coolant having a temperature in the range of 50 to 120° C., and packaged at a temperature below 30° C.

Such solutions, known from the prior art, can offer further potential for improvement. This relates in particular to the provision of NPG compacts of the highest possible purity, which show only a low tendency to caking even under unfavorable storage conditions.

It is the object of the present invention to provide an improved process for obtaining more caking-resistant NPG compacted bodies in the form of compressed compacts, as well as an improved dosage form of the NPG with a reduced tendency to clump during storage.

The task is solved by the features of the independent claims, directed to the process according to the invention as well as the compacts according to the invention. Preferred embodiments of the invention are indicated in the dependent claims, in the description or in the figures, whereby further features described or shown in the dependent claims, in the description or in the figures may individually or in any combination constitute an object of the invention, as long as the context does not clearly indicate the contrary.

In accordance with the invention, the problem is solved by a process for the production of neopentyl glycol compacts, the process comprising at least the process steps of a) providing a fill of NPG flakes; and b) compressing the fill in a mold to form a compact, wherein the compression takes place at a pressure of greater than or equal to 0.5 MPa and less than or equal to 7.5 MPa. Surprisingly, it was found that a variety of different and mechanically stable NPG compacts can be obtained via the process of the invention, which show a significantly reduced tendency to form larger aggregates during a storage or transportation operation compared to NPG flakes. In addition to a lower tendency to caking under different ambient conditions, the compacts that can be produced by means of the process also show a lower tendency to sublimation and, particularly surprisingly, only insignificantly limited solubility in different solvents for large-scale processing. Without being bound by theory, the synergistic advantages of high mechanical strength, low sublimation tendency, lower caking tendency with a relatively good dissolving capacity result from the use of NPG flakes within a mechanical pressing process at relatively low pressures, whereby a mechanically very stable compacted article is obtained which can nevertheless be easily dissolved into its components by solvents. Due to the size distribution of the flakes and their irregular shape, the use of flakes seems to result in the right amount of voids being enclosed in the compact during the pressing process in the specified pressure range, which have only a slight effect on the mechanical strength of the compact, but a very positive effect on the dissolution rate. Higher pressure ranges during pressing tend to increase the mechanical strength only insignificantly, but greatly reduce the dissolution rate in solvents. The latter is probably due to an excessive increase in the compact density, which makes it more difficult for solvents to penetrate the compact. A lower pressing pressure may rather result in an insufficient mechanical strength of the compact for usual storage and transport conditions. In addition, it is further surprising that the compacting process proposed in accordance with the invention also does not change or restrict the desired NPG crystal structure, so that no or only very small heat toning occurs in the pressing process and in the subsequent storage process, which leaves the basic properties of the pressed compact unchanged. This results in a clear advantage over the solutions otherwise proposed in the prior art, since the temperature control in the pressing process can be kept simpler. Lastly and finally, it is particularly advantageous that these property improvements can be realized without the additions of further substances in the NPG compact. The latter solution is particularly disadvantageous, since these further substances either have to be removed at great expense in the further course of the process or their properties have to be taken into account in the formulation of further downstream products, or they can have a negative effect on the quality of the downstream products produced from them.

The process according to the invention is a process for the production of neopentyl glycol compacts. The compacts are molded bodies obtained by compressing material in a compression mold. The pressed articles are characterized by a regular shape, which results from the geometry of the press mold used. For example, spheres, cuboids or briquettes of different shapes and sizes can be produced by the choice of the pressing die. The compacts are shaped substantially the same, so that, for example, different compacts differ in weight by less than 25% by weight, preferably less than 15% by weight, and further preferably less than 10% by weight. The compact is a neopentyl glycol compact in those cases where more than 90 wt %, further more than 95 wt %, and further preferably more than 97 wt % of the compact consists of NPG. The process can be carried out by means of conventional presses in the form of extruder presses, roller presses or by the press chamber method.

The process comprises process step a), in which the provision of a fill of NPG flakes takes place. The NPG compact is produced from a fill of NPG flakes. This fill is a statistical distribution of irregularly broken NPG particles. The NPG flakes have a thickness and size distribution which is a function of the manufacturing process. In addition to the macroscopic flakes, NPG particles may also be present in the bulk in the form of small grains or NPG dust. The amount of dust in the NPG bulk is also a function of the manufacturing process. In terms of quantity, the bulk mainly contains flakes in the form of flat platelets, which have an irregularly broken edge. The size distribution of the NPG flakes can be determined by the mechanical action on the NPG solidified on a cooling belt. The composition of the NPG flakes can be freely selected in certain proportions. Preferably, the NPG flakes can consist of pure NPG. However, it is also possible for the flakes to have other substances as additional components. Preferably, the weight fraction of NPG in the flakes is greater than or equal to 80%, further preferably greater than or equal to 90% and further preferably greater than or equal to 95%.

The process comprises process step b), in which the compression of the fill in a mold to form a compact is carried out. According to the invention, compression takes place at a pressure greater than or equal to 0.5 MPa and less than or equal to 7.5 MPa. The fill of NPG flakes filled into one or more compression molds is subjected to the above pressure range by contacting the two halves of the compression mold. This process can be carried out, for example, by a manual press or continuously by rotating pressing tools. The pressing process can optionally take place within a conditioned environment. However, it is not mandatory that the ambient air or temperature be appropriately preconditioned. More expediently, the pressing process can take place at room temperature. However, it is also possible that, for example, the pressing tools are pre-conditioned to a certain temperature range, for example between greater than or equal to 10° C. and less than or equal to 40° C. The time period for pressing a single mold may vary. For example, the bulk of NPG flakes may remain in the compression mold for greater than or equal to 1 second, further preferably greater than or equal to 2 seconds, and further preferably greater than or equal to 5 seconds. The application of the pressing pressure for a longer period of time is thereby unnecessary. In the course of pressing, in addition to the individual compacts, connecting pieces can also be formed between the compacts, which result from the fact that material from the bulk has also been deposited between the individual pressing dies. This further material does not form the compacts in the sense of the invention and can be sheared off or screened off before further processing of the compacts, for example by applying slight mechanical pressure.

In a preferred embodiment of the process, the NPG flakes can have a bulk density of greater than or equal to 0.5 g/cm³ and less than or equal to 0.6 g/cm³. For the formation of particularly mechanically stable and particularly well-dissolvable compacts, it has been found to be particularly advantageous that the bulk density of the NPG flakes is in the range indicated above. Without being bound by theory, the bulk density in particular also appears to be decisive for the preferred porosity of the compacts. Smaller bulk densities may be disadvantageous, since in these cases the mechanical strength of the compacts may be insufficient. Larger bulk densities, on the other hand, can be disadvantageous, since in these cases the dissolution rate of the compact is reduced too much. The bulk density can be measured, for example, according to DIN ISO 697 or DIN ISO 60.

Within a further preferred embodiment of the process, the NPG flakes may have a fines content of less than or equal to 6 mm, determined by sieving, of greater than or equal to 80% by weight and less than or equal to 90% by weight. It has also been found to be advantageous for the mechanical properties of the compact that the NPG flakes used in the NPG fill satisfy a certain size distribution. In particular, a higher proportion of small flakes, specified here as a fine fraction with a size smaller than 6 mm, can lead to both an improvement of the mechanical strength and the dissolving capacity of the compact.

Within a further preferred aspect of the process, the NPG flakes may have a thickness of greater than or equal to 0.75 mm and less than or equal to 5 mm. In addition to the lateral extent of the NPG flakes, their thickness also has an influence on the achievable strength and the achievable dissolution rate of NPG compacts. The thickness of the flakes can be conveniently determined by the height of the NPG melt on the cooling belt used for production. Smaller thicknesses of the NPG flakes can be disadvantageous, since in these cases the mechanical strength of the obtainable compact can be reduced. Higher thicknesses can be disadvantageous, since in these cases also unfavorable mechanical properties of the compact can be obtained. Without being bound by theory, this relationship can probably be due to the fact that the individual flakes can resist deformation in the press to a greater extent and, in this respect, a reduced interaction between the individual flakes is induced by the pressing process. The thickness of the NPG flakes can be determined, for example, using a caliper gauge. To obtain a statistically validated value about the fill, for example, 50 selected flakes can be measured. Further advantageously, the thickness of the flakes can be greater than or equal to 1 mm and less than or equal to 4 mm, further preferably greater than or equal to 1.5 mm and less than or equal to 3 mm.

According to a preferred characteristic of the process, the NPG flakes can have a D50 quantile, determined via sieving, of greater than or equal to 2 mm and less than or equal to 8 mm. NPG flakes with the above quantile have proven to be particularly suitable for the production of especially mechanically stable and rapidly dissolving compacts. This size range of the NPG flakes with a high proportion of rather smaller particles, together with a relatively low pressing pressure and a relatively short pressing time, can lead to the creation of particularly suitable interactions between the individual particles to be pressed. A very homogeneous compact is formed, which dissolves well in a relatively short time on contact with a solvent.

In a further preferred embodiment of the process, the NPG flakes can have a D95 quantile, determined by sieving, of greater than or equal to 7.5 mm and less than or equal to 10 mm. The use of NPG flakes with a relatively low proportion of flakes above 10 mm can also result in particularly homogeneous compacts being obtainable, which are characterized by only a low proportion of fragments even under greater mechanical stress.

In a further preferred embodiment of the process, compression can be carried out at a pressure of greater than or equal to 1.0 MPa and less than or equal to 4 MPa. The above-mentioned compression pressure has proved to be particularly suitable for obtaining compacts that are as uniform and mechanically stable as possible. Particularly in the range of relatively low compression pressures, sufficient stability and very fast disintegration of the compacts can be achieved.

In a further embodiment of the process, process step b) can be carried out in a temperature range of greater than or equal to 5° C. and less than or equal to 40° C. To obtain particularly uniform compacts and to prevent caking in the dies, the above temperature range has proved to be particularly suitable. Higher temperatures can be disadvantageous, as unwanted thermodynamic phase transformation of the NPG can be induced in these ranges. Furthermore, higher temperatures in the process can lead to unintentional buildup of production material on the walls of the press during the course of continuous production, which is not removed from the mold independently during the pressing processes. Lower temperatures during pressing can be disadvantageous, since in these cases the lower pressing pressure results in insufficient pressing of the individual particles against each other.

Within a further preferred embodiment of the process, the NPG flakes can have a water content of greater than or equal to 0.05 wt. % and less than or equal to 3 wt. %. For the production of particularly uniform compacts, it has been found to be especially advantageous that the NPG flakes have a defined water content. In addition to the influence on the mechanical properties, the water content of the bulk can also exert an influence on the producibility of the compacts. In particular, it may happen that with too high water contents the mechanical press forms have to be cleaned more frequently. The water content of the NPG flakes can be determined by known methods, such as Karl-Fischer.

Furthermore, an NPG compact according to the invention is one in which the compact has a density of greater than or equal to 0.9 g/cm³ and less than or equal to 1.02 g/cm³. By means of the process according to the invention, pressed articles can be obtained which, starting from the NPG used and starting from the use as NPG flakes, have a very small range of specific density. Within this very narrow NPG density range, mechanically very stable compacts are obtained which show only a very slight tendency to caking even under unfavorable storage conditions. In addition to the improved mechanical properties and the low sublimation tendency of the compacts as such, the compacts nevertheless exhibit good solubility in the usual solvents for NPG, such as water. This nevertheless good solubility in comparison with NPG flakes is surprising, since the pressed compacts have a significantly smaller surface area compared with the NPG flakes. In this respect, due to the difference in surface area, the skilled person would expect the dissolution rate of the NPG compacts to be more significantly reduced. The density of the compacts can be determined by methods known to the skilled person, for example by measuring and weighing the compacts.

According to a preferred characteristic of the NPG compact, the compact may have a volume greater than or equal to 2.5 cm³ and less than or equal to 15 cm³. In order to achieve the best possible balance between the dissolution rate of the individual particles and the mechanical stability of the compact, the above-mentioned volumes have proven to be particularly suitable for the individual compact. Smaller compacts may comprise a too large a surface area, which can lead to an excessively high fines content during storage due to increased sublimation from the surfaces of the individual compacts. Larger compact volumes, on the other hand, can be disadvantageous, since in these cases the dissolution rate of the compacts, for example in water, is reduced too much.

In a further preferred embodiment of the NPG compact, the compact may have a density greater than or equal to 0.95 g/cm³ and less than or equal to 1.05 g/cm³. Conditional on the bulk density of the NPG flakes and on the pressing pressure applied in the production, the density of the resulting NPG compacts can be determined. These parameters make it possible to obtain pressed articles with a density in the above-mentioned range. This very narrow range of material density for the NPG compacts can lead in particular to mechanically very stable compacts being obtained, which exhibit particularly rapid dissolution in the solvents suitable for this purpose. In addition, this density range can lead to the final mechanical strength of the compacts being obtained after a relatively short time.

Within a preferred aspect of the NPG compact, the compact may have a surface-to-mass ratio of greater than or equal to 0.4 m² kg and less than or equal to 0.6 m² kg. For NPG compacts in particular, it has been found suitable to use dies with a basic geometry that has the above surface-to-mass ratio. These geometries show only a low degree of mass loss (sublimation) during storage, lead to only minor abrasion on the individual compacts even under heavy mechanical stress, and these compacts can also be processed with standard equipment in an industrial environment. The surface-to-mass ratio can be determined by weighing out and determining the size of the pressed part, for example using a caliper gauge.

In a further preferred embodiment of the NPG compact, the compact may have an NPG content of greater than or equal to 98 wt %. Surprisingly, it has been shown that even without the addition of further substances, for example in the form of disintegrants or in the form of anti-caking agents, mechanically stable moldings can be obtained which show only a very slight tendency to caking during storage or transport. In this respect, it should be particularly emphasized that further processing of the NPG can be carried out without regard to the presence of further substances. In particular, the compacts according to the invention are largely free of further substances. Thus, the NPG content in the compact may be greater than or equal to 98.5% by weight, further preferably greater than or equal to 99.5% by weight, and further preferably greater than or equal to 99.9% by weight. Particularly preferably, the compact may also consist of 100% NPG. The proportion by weight of NPG in the compacts can be quantitatively determined, for example, by HPLC analysis, neglecting the water content.

In a further embodiment of the NPG compact, the compact may have a compressive strength of greater than or equal to 80 N and less than or equal to 400 N. The compacts according to the invention are characterized by a relatively high fracture strength, which, surprisingly, is obtainable via only a relatively low compression pressure as part of the manufacturing process. To obtain consistent values, the breaking strength of the individual compacts is determined 24 hours after manufacture and storage at room temperature. Within this period, the pressed part as such can still post-harden and develop higher fracture strengths. The compressive strength is determined using a compression strength tester from Erichson (Model 469 E4). The upper and lower plates each have a diameter of 80 mm. The diameter of the measuring body is 10 mm (horizontal) and the speed of the measuring body is 8 mm/min.

Within a further preferred embodiment of the NPG compact, the compact may have a cuboid geometry. For handling in industrial packaging and transport processes, it has been found to be particularly suitable that the compacts have a cuboid geometry. This geometry can contribute to a particularly low tendency to caking and to a particularly low level of abrasion, even under heavy mechanical stress during transport. The compact can have an exact cuboid geometry or also a geometry which is based on a cuboid. Thus, via this definition, briquettes are understood in the usual sense as well as egg coals or egg briquettes, which have a basic cuboid geometry with rounded corners. The cuboid geometry may also be a cube. In addition to the basic geometry, the pressed briquettes may also have a, for example, circumferential, pressed seam and other features, such as a logo or the like on the surface.

Within a further preferred aspect of the NPG compact, the ratio of the averaged length and width to the height of the cuboid compact, determined by (length+width)/2 divided by the height, can be greater than or equal to 1.25 and less than or equal to 3.5. Due to the fact that in industrial production the compacts are not neatly layered, but are provided in a disordered bulk in bags or big-bags, the above ratio of height and width of the cuboid compact has been found to be particularly suitable. This aspect ratio of the compact can result in only a small amount of breakage being obtained in the compact bulk, even under unfavorable storage conditions and under high mechanical stress. In addition, due to the specified asymmetry of the cuboidal compact, a suitable dissolution rate of the compact in the usual solvents can also be achieved.

Furthermore, according to the invention, bulk materials made of caking-resistant neopentyl glycol compacts are used, whereby the proportion of fine particles <1 mm in the bulk materials is below 10%. These bulk materials can be characterized by a particularly low proportion of caking, even under unfavorable storage and transport conditions.

### EXAMPLES

For the production of NPG compacts, a bulk of NPG flakes is compressed in a roller press into compacts of different sizes in the form of NPG briquettes. The NPG flakes consist of 100% NPG (neglecting the water content). No additional anti-caking, binding or disintegrating agents are added to the NPG flakes or to the NPG as such. The flakes were analyzed by sieve analysis with the following size classes (data in mm): >20, 20-10; 10-8; 8-6.3; 6.3-5; 5-4; 4-3.15; 3.15-2; 2-1; 1-0.5; 0.5-0.25; 0.25-0.125; and 0.125-0 mm. This results in a D50 quantile of 3.45 mm and a D95 quantile of 8.7 mm. The density of the NPG fill is 0.525 g/cm³.

Two different sizes of briquettes are produced, with the basic symmetry of the press forms corresponding to an egg coal briquette basic form. The dimensions of the briquettes are as follows:

| Nominal volume [cm³] | Length [mm] | Width [mm] | Height [mm] |
|---|---|---|---|
| 5 | 30 | 24 | 17 |
| 10 | 33 | 30 | 20 |

For both sizes, pressing is carried out at a pressure of 1 MPa. The briquettes are then sieved to separate the fines (particles smaller than 6.3 mm). Without fines, the yield of the pressing process is over 90%. The briquettes are stored for 24 h at room temperature and a relative humidity of 85%. Under these storage conditions, no change in briquette mass due to hygroscopic behavior was observed. The compressive strength of the pressed compacts is significantly improved by the 24 h storage. Directly after pressing, the compressive strength determined by a compression strength measurement (“Compression Strength of Briquettes”, measured with a Type 469 ERICHSEN machine) is >100 N. After storage for 24 h under the above conditions, the compressive strength can be further increased. Typical compressive strength values after storage are 109 N for the smaller briquette and 155 N for the larger briquettes. The briquettes also pass a drop test from a height of 2 m. After 2 runs on unstored briquettes, approximately 90% intact briquettes were still found for both briquette sizes. These results of the drop tests also improved after 24 h storage of the freshly produced briquettes.

To determine the mass loss of the compacts due to sublimation during storage, the briquettes are placed in a fume hood and the mass loss is followed over 49 days. The briquettes show a linear sublimation behavior, with the mass loss after 49 days being about 10% for the 10 cm³ compact and 11% for the 5 cm³ compact. In contrast, the mass loss of NPG flakes under these conditions is about 18%.

In addition, tests are carried out on compact hygroscopy under defined humidity in a desiccator. For this purpose, the briquettes are placed in an open tray in a desiccator containing various saturated salt solutions. Saturated NaCl solution provides a relative humidity of 74%, whereas a lower humidity of 11% is obtained by using saturated LiCl solution. The weight and water content of the pressed compacts are noted after two weeks. By storing at a relative humidity of 11%, no significant increase in water content or loss of mass of the compacts can be observed. When stored under 74% humidity, a water content of 1.3% (10 cm³ briquettes) and 1.6% (5 cm³ compact) was measured after 2 weeks. Thus, the pressed compact show a significantly lower hygroscopy compared to NPG flakes.

Storage tests were also carried out on the compacts under pressure. For this purpose, a glass cylinder (diameter 15.3 cm) with an autoclave insert as weight (14.3 kg) is used in each case with a suitable Teflon disc in between. This setup provides a total storage pressure of 78 g/cm² and corresponds approximately to the pressure prevailing in the lowest bag of a stack of bags or in the lower area of a big bag. The tests are carried out in each case with a bulk height of 10 cm at room temperature. A clear difference in the storage behavior of the pressed compacts compared to the NPG flakes can be observed. After one week of storage, only a minimal portion of the material is caked on the surface in the case of the pressed compacts. NPG flakes show a significantly higher proportion of caking and agglomeration under these conditions. Even adhering briquettes can be separated again very easily by applying low forces. After 4 weeks of storage, a similar picture emerges for the pressed briquettes compared to the NPG flakes. The degree of adhesion increases for both the NPG flakes and the pressed compacts, yet the proportion of caked material is clear for the flakes. In addition, adhered compacts can be easily detached from each other by applying small forces. The adhering NPG flakes cannot be completely detached from each other even by applying higher forces.

To compare the dissolution behavior between NPG flakes and compacts, a 25 wt % NPG solution is prepared in water with stirring in each case. Surprisingly, the dissolution behavior of the briquettes is practically independent of the pressed compact size used. The dissolution rate of the compacts is about 12:20 min for the 5 cm³ and 12:40 min for the 10 cm³ compacts. NPG flakes dissolve in about 2:53 min under the same experimental conditions. Considering the difference in surface area available for dissolution of the flakes and the compacts, it can be estimated that the surface area of the flakes is higher by a factor of at least 10 compared to the compacts. The flake surface area can be approximated by assuming cylindrical particles with the flake height used as the height and a diameter corresponding to the D50 quantile of the flakes. This approach does not take into account the nevertheless considerable fraction of very small fragments in the flakes and leads to a very conservative estimate of the flake surface area. Compared to the flakes, the compacts dissolve worse by a factor of 4-5, whereas the difference in the factor with respect to the available surface area is in any case higher than 10. In this respect, the pressed compacts dissolve much better than could be expected. Without being bound by theory, this is attributed to the specific density of the compacts, which indicates that there is no compact NPG compact. Due to the use of NPG flakes and the application of only low pressing pressures, the compacts also contain a significant amount of pores or air in the compact, which apparently facilitates the diffusion of solvents and thus the dissolution.

Some typical parameters of the compacts according to the invention obtainable by the process according to the invention are as follows:

| | |
|---|---|
| 5 cm³ | 10 cm³ |

| | | | | |
|---|---|---|---|---|
| Dimensions | 30 × 24 × 10 | mm | 33 × 30 × 20 | mm |
| L × W × H | | | | |
| Weight | 10 | g | 13-15 | g |
| Surface | 0.0049 | m² | 0.0068 | m² |
| Surface/mass | 0.49 | m²/kg | 0.49 | m²/kg |
| Bulk density | 0.535 | g/cm³ | 0.531 | g/cm³ |
| Density | 0.97-0.99 | g/cm³ | 0.97-0.99 | g/cm³ |

| | | |
|---|---|---|
| Fine fraction | <10% | <10% |
| (<6 mm) | | |
| Mass loss | 0.22 wt %/24 h | 0.2 wt %/24 h |
| | 1.6 wt %/week | 1.4 wt %/week |

1. A process for the production of neopentyl glycol compacts, wherein the process comprises at least the process steps:
a) Provide a fill of NPG flakes;
(b) compressing the fill in a mold to form a compact, wherein the compressing is performed at a pressure greater than or equal to 0.5 MPa and less than or equal to 7.5 MPa.

2. The process of claim 1, wherein the NPG flakes have a bulk density greater than or equal to 0.5 g/cm³ and less than or equal to 0.6 g/cm³.

3. The process of claim 1, wherein the NPG flakes have a fines content of less than or equal to 6 mm, as determined by sieving, of greater than or equal to 80 wt % and less than or equal to 90 wt %.

4. The process of claim 1, wherein the NPG flakes have a thickness of greater than or equal to 0.75 mm and less than or equal to 5 mm.

5. The process of claim 1, wherein the NPG flakes have a D50 quantile, determined via sieving, of greater than or equal to 2 mm and less than or equal to 8 mm.

6. The process of claim 1, wherein the NPG flakes have a D95 quantile, determined by sieving, of greater than or equal to 7.5 mm and less than or equal to 10 mm.

7. The process of claim 1, wherein the compressing is performed at a pressure greater than or equal to 1.0 MPa and less than or equal to 4 MPa.

8. The process according to claim 1, wherein the process step b) is carried out in a temperature range of greater than or equal to 5° C. and less than or equal to 40° C.

9. The process according to claim 1, wherein the NPG flakes have a water content of greater than or equal to 0.05 wt % and less than or equal to 3 wt %.

10. A NPG compact, wherein the compact has a density greater than or equal to 0.9 g/cm³ and less than or equal to 1.02 g/cm³.

11. The NPG compact according to claim 10, wherein the compact has a volume greater than or equal to 2.5 cm³ and less than or equal to 15 cm³.

12. The NPG compact according to claim 10, wherein the compact has a density greater than or equal to 0.95 g/cm³ and less than or equal to 1.02 g/cm³.

13. The NPG compact according claim 10, wherein the compact has a surface-to-mass ratio of greater than or equal to 0.4 m²/kg and less than or equal to 0.6 m²/kg.

14. The NPG compact according to claim 10, wherein the compact has an NPG content greater than or equal to 98 wt %.

15. The NPG compact according to claim 10, wherein the compact has a compressive strength of greater than or equal to 80 N and less than or equal to 400 N.
